(19) United States
(12) Patent Application Publication
(10) Pub. No.: US 2011/0042247 A1
(43) Pub. Date: Feb. 24, 2011

(54) FORMULATIONS OF AZACITIDINE AND ITS DERIVATIVES

(76) Inventors: Chandrasekhar Kocherlakota, Secunderabad (IN); Nagaraju Banda, Hyderabad (IN); Tarun Singh, Bijnor (IN); Prahlad Pragallapati, Eluru (IN); Sachin Sharma, Kangra (IN); Raghu Nandan Srinivasan, Bangalore (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/823,184

(22) Filed: Jun. 25, 2010

Related U.S. Application Data
(60) Provisional application No. 61/293,002, filed on Jan. 7, 2010.

(30) Foreign Application Priority Data
Jun. 25, 2009 (IN) 1485/CHE/2009

(51) Int. Cl. B65D 69/00; A61K 31/706; A61P 35/00; A61B 19/02

(52) U.S. Cl. 206/223; 514/43; 206/438

(57) ABSTRACT

The present invention relates to pharmaceutical formulations comprising azacitidine or its pharmaceutically acceptable salts, including processes for preparing the formulations comprising azacitidine, or salts thereof, and methods of using the formulations for treating various cancer disorders in mammals.

Aspects of the present invention relate to pharmaceutical formulations comprising azacitidine or pharmaceutically acceptable derivatives thereof. Further aspects of the invention relate to process for preparing stable formulations comprising azacitidine or its derivatives, and methods of using the formulations for treating various types of cancer disorders in mammals.

The drug compound having adopted names “azacitidine” (INN) and “5-azacytidine” is a pyrimidine nucleoside analog of cytidine. It is an azacytosine nucleoside, present in DNA and RNA. Chemical names for azacitidine are 4-amino-1-β-D-ribofuranosyl-1,3,5-triazin-2(1H)-one, and 4-amino-1-β-D-ribofuranosyl-s-triazin-2(1H)-one, and the compound is represented by structural Formula 1.

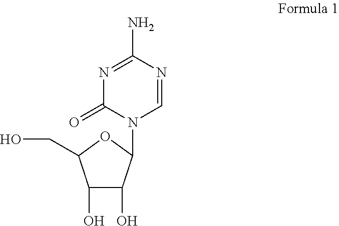

Azacitidine is insoluble in acetone, ethanol, and methyl ethyl ketone, slightly soluble in ethanol and water (50:50), propylene glycol, and polyethylene glycol, sparingly soluble in water, water saturated octanol, 5% dextrose in water, N-methylpyrrolidone, normal saline, and 5% Tween™ 80 in water, and soluble in dimethylsulphoxide (DMSO).

Azacitidine and its deoxy derivative, decitabine (also known as 5-aza-2′-deoxycytidine), are used in the treatment of myelodysplastic syndrome. Decitabine, an analogue of the natural nucleoside 2′-deoxycytidine. Decitabine is fine, white to almost white powder with the molecular weight of 228.21. Decitabine is slightly soluble in ethanol/water (50/50), methanol/water (50/50) and methanol, sparingly soluble in water and soluble in dimethyl sulphoxide (DMSO). Its chemical name is 4-amino-1-(2-deoxy-β-D-erythro-pentofuranosyl)-1,3,5-triazin-2(1H)-one and it has the following structural Formula II.

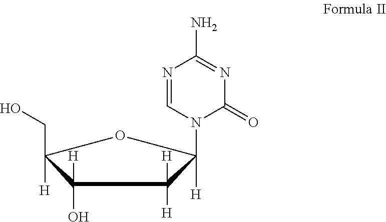

A commercially available product containing azacitidine is sold as VIDAZA® azacitidine for injection, by Celgene. The VIDAZA product received marketing approval in the U.S. in 2004 and is supplied in a sterile form for reconstitution as a suspension for subcutaneous injection, or reconstitution as a solution with further dilution for intravenous infusion. Vials of the VIDAZA product contain 100 mg of azacitidine and 100 mg of mannitol, as a sterile lyophilized powder.

A commercially available product containing decitabine is sold as DACOGEN™ by MGI Pharma. The DACOGEN product is for injection and is available as a white to almost while lyophilized sterilized powder supplied in a glass vial. Each 20 mL single dose glass vial contains decitabine, monobasic potassium phosphate, and sodium hydroxide. Each DACOGEN vial is to be aseptically reconstituted with 10 mL of sterile water for injection, and upon reconstitution each mL contains approximately 5 mg of decitabine at pH 6.7-7.3. Immediately after reconstitution, the solution should be further diluted with 0.9% of sodium chloride injection, 5% dextrose injection, or lactated Ringer injection, to a final concentration of 0.1-1.0 mg/mL.

Azacitidine is believed to exert its antineoplastic effects by causing hypomethylation of DNA and direct cytotoxicity on abnormal hematopoietic cells in the bone marrow. Hypomethylation may restore normal functions to genes that are critical for differentiation and proliferation. The cytotoxic effects of azacitidine cause the death of rapidly dividing cells, including cancer cells that are no longer responsive to normal growth control mechanism. Non-proliferating cells are relatively insensitive to azacitidine.

U.S. Pat. No. 4,684,630 discloses a method of parenterally delivering the aqueous-unstable 5-azacytosine arabinoside and 5-azacytidine compounds, involving an aqueous dilution of a stable, anhydrous organic solution having the drug dissolved therein. The resulting organic-aqueous solution is physiologically suitable for parenteral delivery into a warm-blooded mammal and contains the drug in an effective dosage concentration per unit volume.

U.S. Pat. No. 4,983,586 relates to aqueous parenteral solutions of drugs that are insoluble or only sparingly soluble in water, and/or unstable in water, the aqueous solutions containing hydroxypropyl-beta-cyclodextrin, to provide a means for alleviating problems associated with drug precipitation at the injection site and/or in the lungs or other organs following parenteral administration.

U.S. Pat. No. 6,943,249 relates to methods for isolating crystalline polymorphic Form I of 5-azacytidine, substantially free of other forms, and compositions thereof.

U.S. Patent Application Publication No. 2006/0128654 relates to pharmaceutical formulations of cytidine analogs and derivatives, such as 5-azacytidine, 5-aza-2′-deoxy-2′,2′-difluorocytidine, 5-aza-2′-deoxy-2′-fluorocytidine, 2′-deoxy-2′,2′-difluorocytidine, and cytosine 1-β-D-arabinofuranoside, as well as methods of manufacturing the formulations. In particular, a cytidine analog or derivative is formulated with a cyclodextrin compound to stabilize and/or enhance solubility of the drug. Kits and methods for using the pharmaceutical formulations are also provided, including methods of administering the cytidine analog or derivative to treat conditions or diseases, such as cancer and hematological disorders.

U.S. Patent Application Publication No. 2006/0063735 relates to salts of 5-azacytidine as well as methods for synthesizing the salts. Pharmaceutical compositions and methods of using the 5-azacytidine salts are also provided, including methods of administering the salts or pharmaceutical compositions thereof to treat conditions such as cancer and hematological disorders.

The literature reports that VIDAZA, reconstituted with 4 mL of sterile water for injection to form a suspension for subcutaneous administration, may be stored for up to 1 hour at 25° C., or for up to 8 hours between 2° C. and 8° C. VIDAZA reconstituted with 10 mL of sterile water for injection for intravenous administration may be stored at 25° C., but the administration must be completed within 1 hour after reconstitution. The duration of IV infusion administration is limited by the decomposition and instability of azacitidine, and low aqueous solubility of the drug in aqueous solutions. Further azacitidine hydrolyzes quickly in water, converting into other forms.

Stable compositions that overcome problems associated with VIDAZA or DACOGEN products are needed.

### SUMMARY

Aspects of the present invention relate to pharmaceutical formulations containing azacitidine or its pharmaceutically acceptable salts. Further aspects of the invention relate to processes for preparing stable formulations comprising azacitidine or salts thereof, and methods of using the formulations for treating various types of cancer disorders in mammals.

In an aspect, the invention includes processes for preparing stable formulations comprising azacitidine or its salts.

The invention also relates to processes for preparing formulations containing azacitidine or a salt thereof, wherein embodiments comprise:

a) preparing an aqueous solution of azacitidine at about −3° C. to −1° C.; and

b) lyophilizing the solution.

An aspect of the present invention provides solid formulations containing azacitidine, wherein azacitidine retains its physical form after storage at 25° C. and 60% relative humidity for at least 2 weeks.

An aspect of the present invention provides solid formulations containing azacitidine, wherein azacitidine retains its physical form during storage at 2-8° C. for at least 2 weeks.

In embodiments, the invention includes solid formulations containing azacitidine, having water content less than about 6% by weight.

In embodiments, the invention relates to formulations containing azacitidine, wherein the content of a 1-β-D-ribofuranosyl-3-guanylurea (RGU) impurity is less than about 1% of the label azacitidine content.

In embodiments, the invention relates to formulations containing azacitidine, wherein the content of a N-(formylamidino)N′-β-D-ribofuranosylurea (RGU-CHO) impurity is less than about 5% of the label azacitidine content.

In embodiments, the invention relates to formulations containing azacitidine, wherein total drug-related impurities, excluding N-(formylamidino) N′-β-D-ribofuranosylurea (RGU-CHO), are less than about 2% of the label azacitidine content.

### BRIEF DESCRIPTION OF THE DRAWING

FIG. 1 shows powder X-ray diffraction patterns of the formulation prepared in Example 1 (“A”), the formulation after storage at 2-8° C. for 2 weeks (“B”), and the formulation after storage at 25° C. and 60% relative humidity for 2 weeks (“C”). The powder X-ray diffraction pattern of a similarly prepared placebo formulation (without any of the active ingredient azacitidine) of Example 1 is shown as “P,” and the powder X-ray diffraction pattern, of the active ingredient azacitidine is shown as “D.”

### DETAILED DESCRIPTION

Aspects of the present invention relate to pharmaceutical formulations comprising azacitidine or pharmaceutically acceptable salts thereof. Further aspects of the invention relate to processes of preparing formulations comprising azacitidine or its salts thereof and methods of using the formulations for treating various cancerous disease conditions.

Azacitidine may be used in the form of a salt, which salt is prepared by reaction of the free base with an inorganic acid or an organic acid. Examples of useful inorganic acids are hydrochloric, hydrobromic, hydroiodic, nitric, carbonic, sulfuric, and phosphoric acids. Appropriate organic acids include aliphatic, cycloaliphatic, aromatic, arylaliphatic, heterocyclic, carboxylic and sulfonic classes of organic acids, specific examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acids. In certain embodiments, the acid is one of hydrochloric, L-lactic, acetic, phosphoric, (+)-L-tartaric, citric, propionic, butyric, hexanoic, L-aspartic, L-glutamic, succinic, EDTA, maleic, methanesulfonic, hydrobromic, hydrofluoric, hydroiodic, nitric, nitrous, sulfuric, sulfurous, phosphorous, perchloric, chloric, chlorous, sulfonic; ascorbic, carbonic, and fumaric acids. In particular embodiments, the acid is one of ethanesulfonic, 2-hydroxyethanesulfonic, and toluenesulfonic acids.

Stability, as referred to in the context of the present invention refers to both physical and chemical stability.

The term “formulation” as used in the context of the present invention refers to any of various dosage forms suitable for administration of a drug, such as parenterally, intraperitoneally, intravenously, intraarterially, intramuscularly, subcutaneously, etc.

The term “pharmaceutically acceptable” refers to an ingredient that is useful in preparing a pharmaceutical composition that is generally safe, non-toxic, and neither biologically nor otherwise undesirable, and includes those acceptable for veterinary use as well as human pharmaceutical use.

The term “azacitidine” is intended to include the free base, as well as salts, polymorphs, isomers, solvates, enantiomers, hydrates, prodrugs, and any mixtures thereof.

The term “derivative” is intended to use any derivatives of azacitidine such as the deoxyderivative, decitabine (also known as 5-aza-2′-deoxycytidine).

The term “physical form” as used in the specification refers to a polymorphic form of azacitidine or its salts, either crystalline or amorphous. A particular polymorphic form can be identified by its powder X-ray diffraction pattern, as well as by other analytical techniques.

The term “physical stability” as used in the specification refers to retaining an original physical form in a formulation, even after storage under any of various stability testing conditions.

The term “chemical stability” as referred in the specification relates to maintaining an original drug purity of the formulation, in terms of drug-related impurities or drug-related substances.

Injectable formulations are typically formulated as aqueous solutions in which water is the primary excipient. Injectable formulations can be prepared in conventional forms, either as liquid solutions or suspensions, solid forms suitable for solubilization or suspending in liquid prior to injection, or as emulsions. Sterile injectable formulations can be prepared according to techniques known in the art using suitable carriers, dispersing or wetting agents, and/or suspending agents. The injectable formulations may be sterile injectable solutions or suspensions in a nontoxic, parenterally acceptable diluent or solvent. Among the acceptable vehicles and solvents that may be employed are water, Ringer's solution and isotonic sodium chloride solution. In addition, fixed oils, fatty esters or polyols are conventionally employed as solvents or suspending media.

The formulations of the present invention are particularly suited for use in parenteral administration, but it will be understood that the solutions may have alternative uses. For example, they may be used as intermediates in the preparation of other pharmaceutical dosage forms.

In an embodiment, the invention includes pharmaceutical formulations comprising azacitidine or its salts and at least one pharmaceutical acceptable excipient.

Azacitidine hydrolyzes quickly in water, and this is dependent on pH and temperature. It has been observed that, due to hydrolysis, around nine solid state forms have been identified: five polymorphic forms, three psueodpolymorphic forms and an amorphous form. Polymorphism could be of importance since speed of dissolution of azacitidine could affect its degradation. Azacitidine rapidly degrades in aqueous solutions via hydrolysis, and due to this instability a lyophilized dosage form was developed to minimize water activity in the dosage form. Hence, the water content of a formulation may impact the stability of the product.

Two major degradants have been observed due to hydrolysis. The hydrolytic pathway leads to the formation of an initial N-formyl compound hydrolysis product “RGU-CHO,” which is a reversible reaction and the compounds are in equilibrium with each other. This is followed by ring opening and loss of formic acid which results in formation of an amine compound “RGU,” which is an irreversible reaction. RGU-CHO is N-(formylamidino) N′-β-D-ribofuranosylurea (“N-formyl compound” below) and RGU is 1-β-D-ribofuranosyl-3-guanylurea (“amine compound” below).

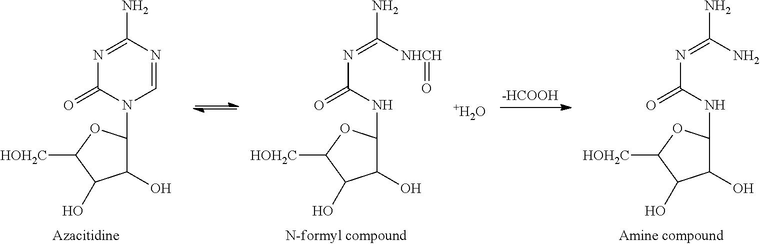

Aspects of the invention provide compositions and pharmaceutical formulations comprising azacitidine or salts thereof, having certain maximum levels of certain impurities.

In embodiments, the invention includes formulations containing azacitidine wherein the content of the 1-β-D-ribofuranosyl-3-guanylurea (RGU) impurity is less than about 1% of the label azacitidine content.

In embodiments, the invention includes formulations containing azacitidine wherein the content of the N-(formylamidino)N′-β-D-ribofuranosylurea (RGU-CHO) impurity is less than about 5% of the label azacitidine content.

In embodiments, the invention includes formulations containing azacitidine, wherein total drug-related impurities, excluding N-(formylamidino) N′-β-D-ribofuranosylurea (RGU-CHO), are less than about 2% of the label azacitidine content.

In an aspect, the invention includes analytical methods to measure the content of impurities in a formulation.

Conditions for a high performance liquid chromatography (“HPLC”) analytical method for determining azacitidine and impurities, except the RGU-CHO impurity, are given below.

Buffer preparation: dissolve 2.84 g of disodium hydrogen phosphate and 2.72 g of potassium dihydrogen phosphate in 1000 mL of Milli-Q Water, adjust the pH to 6.5 with dilute orthophosphoric acid, filter through a 0.22 μm membrane filter, and degas.

Mobile phase A: Buffer.

Mobile Phase B: Mix 600 mL of water and 400 mL of acetonitrile, and degas.

Diluent: Milli Q water having temperatures about 2-8° C.

### Chromatographic System:

Column: Hichrom INODS-3, 250×4.6 mm, 5 μm or equivalent.

Flow rate: 0.8 mL/minute.

Detector wavelength: 210 nm.

Column temperature: 25° C.

Sample cooler temperature: 5° C.

Run time: 60 minutes.

Sample preparation: Reconstitute the contents of one vial with 10 ml of cold water and transfer into a 100 ml volumetric flask. Make up the volume with diluent.

Injection Volume: 10 μL.

Gradient Program:

| Minutes | Mobile Phase A (%) | Mobile Phase B (%) |
|---|---|---|
| 0 | 100 | 0 |
| 8 | 100 | 0 |
| 35 | 75 | 25 |
| 45 | 75 | 25 |
| 46 | 100 | 0 |
| 60 | 100 | 0 |

For the RGU-CHO impurity, conditions for a normal phase HPLC method are described below.

Chromatographic Conditions:

Mobile Phase: Mix n-hexane and ethanol in the volume ratio of 50:50 and degas.

Diluent: Mix dimethylsulphoxide and ethanol in the volume ratio of 20:80.

Column: Chiral Pack IA, 250×4.6 mm, 5 μm or equivalent.

Flow rate: 1.3 mL/minute.

Detector wavelength: 242 nm.

Column temperature: 25° C.

Run time: 25 minutes.

Sample preparation: Reconstitute the contents of one vial with 10 mL of dimethylsulphoxide (DMSO) and transfer to a 50 mL volumetric flask. Rinse the vial with ethanol and transfer to the flask. Finally, make up the volume with ethanol.

Injection volume: 10 μL.

Typical values for relative retention time (RRT, where azacitidine=1), limit of detection (LOD), and limit of quantification (LOQ) of certain impurities from the methods above are tabulated below. The limits are percentages of the azacitidine content.

| Impurity | RRT | LOQ | LOD |
|---|---|---|---|
| 1-β-D-ribofuranosyl-3-guanylurea (RGU) | 0.26 | 0.1 | 0.03 |
| N-(formylamidino)-N′-β-D- | 1.6 | 0.1 | 0.03 |
| ribofuranosylurea (RGU-CHO) | | | |

In embodiments, the invention includes pharmaceutical formulations comprising azacitidine, wherein the formulations are ready-to-use.

In embodiments, the invention includes pharmaceutical formulations comprising azacitidine, wherein the formulations are reconstitutable formulations.

In embodiments, the invention includes pharmaceutical formulations comprising azacitidine which are either ready-to-use formulations, or reconstitutable formulations, wherein the formulations may be in the form of dry powders, solutions, dispersions, emulsions, or suspensions.

Aspects of the present invention relate to processes for preparing ready-to-use formulations and also relate to processes for preparing reconstitutable formulations.

In embodiments, the invention includes stable pharmaceutical formulations of azacitidine or its salts, together with at least one pharmaceutically acceptable excipient.

In aspects, the invention includes processes for preparing stable formulations comprising azacitidine or its salts.

In embodiments, the invention includes conditions for lyophilizing azacitidine-containing compositions.

Aspects of the invention relate to processes for preparing stable formulations of azacitidine or its salts, wherein embodiments comprising:

a) preparing an aqueous solution containing azacitidine at −3° C. to −1° C.; and

b) lyophilizing the solution.

Embodiments further include storing the solution at temperatures below about 10° C., or about −3° C. to about −1° C., prior to lyophilizing.

In an aspect the invention includes stable formulations of azacitidine having water content less than about 6% by weight.

In embodiments, the pharmaceutical formulations maintain at least about 80%, 90%, 95%, or more of their initial azacitidine content, during storage in a closed container for at least two weeks at any one or more of the following conditions: about 2-8° C.; about 25° C. and 60% relative humidity (RH); about 40° C. and 75% RH; and about 60° C.

The present invention further relates to physical properties for the compositions and pharmaceutical formulations comprising azacitidine or its salts, wherein the properties include, but are not limited to, porosity, density, particle size, dispersibility, moisture content, pH, syringability, injectability, particulate matter, and endotoxins.

In an aspect of the invention, a sterile vessel is provided, containing azacitidine for administration to a subject in need thereof. In embodiments, a sterile vessel comprises a pharmaceutical formulation according to the present invention. The vessel, for example, may be a vial, syringe, or ampoule.

Aspects of the invention also relate to compositions and pharmaceutical formulations in lyophilized form, wherein the compositions after reconstitution form a clear solution with 3 minutes or less of gentle agitation.

The technique known as lyophilization is sometimes employed to process injectable pharmaceuticals that exhibit poor active ingredient stability in aqueous solutions. Lyophilization processing is suitable for injectables because it can be conducted under sterile conditions, which is a primary requirement for parenteral dosage forms. Cryoprotectants are excipients whose primary function is to protect the active constituent during a freezing process. Cryoprotectants in the present invention include bulking agents that may be used in the invention.

Lyophilization or freeze-drying is a process in which water is removed from a product after it is frozen and placed under a vacuum, allowing the ice to change directly from a solid to a vapor, without passing through a liquid phase. The process consists of three separate, unique, and interdependent processes; a freezing phase, a primary drying phase (sublimation), and a secondary drying phase (desorption). These processes may be optimized to enhance the product stability as well as decrease the manufacturing costs.

### Freezing Phase:

A primary function of the freezing phase is to ensure that the entire container having the complex solution is completely frozen, prior to proceeding to a subsequent phase. Additionally, it is usually desired that these containers freeze in a uniform manner. While there are different ways that this can be accomplished, one option is to chill the containers after they are loaded onto the lyophilizer shelves and holding for 30-60 minutes prior to initiation of the freezing cycle. It is generally not practical to equilibrate the shelves to a freezing temperature, because of frost accumulation during the filling and loading of the containers.

### Primary Drying Phase:

Once the formulation is brought to the desired frozen state, primary drying via sublimation can proceed. The primary drying phase involves the removal of bulk water at a product temperature below the ice transition temperature under a vacuum (pressures typically between 50-300 mTorr). This phase can be a critical one for stabilizing an active. The goal is to identify the glass transition temperature (Tg′) for the formulation. The Tg′ is the temperature at which there is a reversible change of state between a viscous liquid and a rigid, amorphous glassy state. One can measure the Tg′ of candidate formulations using a differential scanning calorimeter (DSC), in particular with modulated DSC. Generally, the collapse temperature is observed to be about 2-5° C. greater than the Tg′. Hence, the shelf temperature is set such that the target product temperature is maintained near or below the Tg′ of the formulation throughout the removal of solvent during the primary dry phase.

As the solvent is progressively removed from the formulation containers, the product temperature will approach and reach the shelf temperature since it is no longer cooled by water sublimation. To optimize the duration of the primary dry phase, the removal of solvent vapor can be tracked using a moisture detector, or by monitoring the decrease in pressure difference between a capacitance manometer and a thermocouple pressure gauge or by a pressure drop measurement. The optimization of the primary dry cycle involves a removal of solvent as quickly as possible without causing cake collapse and subsequent product instability.

### Secondary Drying Phase:

The secondary drying phase is the final segment of the lyophilization cycle, where residual moisture is removed from a formulation's interstitial matrix by desorption with elevated temperatures and/or reduced pressures. The final moisture content of a lyophilized formulation, which can be measured by Karl Fischer or other methods, is important because if the solid cake contains too much residual moisture, the stability of the active can be compromised. Hence, it is imperative that one achieves a moisture level as low as possible.

To accomplish a low residual moisture, the shelf temperature is typically elevated to accelerate desorption of water molecules. The duration of the secondary drying phase is usually short. When microstructure collapse occurs, the residual moisture is generally significantly greater than desired. One alternative is to purge the sample chamber of the lyophilizer with alternating cycles of an inert gas such as nitrogen, to facilitate displacement of bound water. However, another solution is to properly formulate the drug product and run an optimal lyophilization cycle.

The advantages of lyophilization include: ease of processing a liquid, which simplifies aseptic handling; enhanced stability of a dry powder; removal of water without excessive heating of the product; enhanced product stability in a dry state; and rapid and easy dissolution of reconstituted product. The product is dried without elevated temperatures, thereby eliminating adverse thermal effects, and then stored in the dry state in which there are relatively few stability problems.

Additionally, freeze dried products are often more soluble, dispersions are stabilized, and products subject to degradation by oxidation or hydrolysis are protected.

An embodiment of a lyophilization process includes the following steps:

1) maintain solvent at temperatures ranging from about −1° C. to −3° C.;

2) add one or more suitable excipients to the step 1) solution;

3) add drug to the step 2) mixture and stir until it completely dissolves;

4) make up volume to a desired quantity with solvent at temperatures of about −1° C. to −3° C.;

5) filter the solution through a sterilizing filter, fill into a container and loosely cover the container;

7) load covered containers into a lyophilizer with precooled shelves; and

8) lyophilize the containers.

Pharmaceuticals to be freeze dried are frequently in aqueous solutions, ranging from about 0.01 to 40% by weight concentrations of total solids. Usually, an improvement in stability of the lyophilizate, compared to a solution, is due to the absence of water in the lyophilizate.

A pharmacologically active constituent of many pharmaceutical products is present in such small quantities that, if freeze dried alone, it may not give a composition of suitable mass, and in some cases its presence would be hard to detect visually. Therefore, excipients are often added to increase the amount of solids present. In most applications it is desirable for a dried product cake to occupy essentially the same volume as that of the original solution. To achieve this, the total solids content of the original solution is frequently about 10 to 25% by weight.

Bulking substances that are useful for this purpose, often in combination, include, but are not limited to, sodium or potassium phosphates (monobasic potassium phosphate, potassium dihydrogen phosphate, etc.), citric acid, tartaric acid, gelatin, lactose and other carbohydrates such as dextrose, mannitol and dextran, and occasionally preservatives. Various excipients contribute appearance characteristics to the cake, such as dull and spongy, sparkling and crystalline, firm or friable, expanded or shrunken, and uniform or striated. Therefore formulations of a composition to be freeze dried should be a result of consideration not only of the nature and stability characteristics required during the liquid state, both freshly prepared and when reconstituted before use, but also the characteristics desired in the final lyophilized cake.

The injectable pharmaceutical formulations may optionally include one or more other pharmaceutically acceptable excipients. The pharmaceutically acceptable excipients may include any one or more of: antibacterial preservatives, such as one or more of phenylmercuric nitrate, thiomersal, benzalkonium chloride, benzethonium chloride, phenol, cresol, and chlorobutanol; antioxidants including one or more of ascorbic acid, sodium sulfite, sodium bisulfite and sodium metabisulfite; chelating agents such as ethylenediamine tetraacetic acid (EDTA); buffers including one or more of acetate, citrate, tartarate, phosphate, benzoate and bicarbonate buffers; tonicity contributors including one or more of sodium chloride, potassium chloride, dextrose, mannitol, sorbitol and lactose; and alkaline substances including one or more of sodium hydroxide, potassium hydroxide, sodium carbonate and meglumine.

In aspects the invention includes kits provided for delivery of the azacitidine or its salts. A kit according to the present invention comprises a container holding the drug composition, a sterile reconstitution vehicle, and a sterile syringe.

The area used for processing the compositions and formulations of the present invention generally should comply with the requirements given in the current United States Pharmacopoeia (“USP”) for parenteral dosage forms.

In embodiments, the active azacitidine may be replaced with other active ingredients in the same therapeutic class, such as decitabine (also known as 5-aza-2′-deoxycytidine, a derivative of azacitidine).

Certain specific aspects and embodiments of the invention will be further described in the following examples, which are provided only for purposes of illustration and are not intended to limit the scope of the invention in any manner.

### Example 1

### Azacitidine Pharmaceutical Formulation

| Ingredient | mg/Vial | |
|---|---|---|
| Azacitidine | 100 | |
| Mannitol | 100 | |
| Water* | q.s. to 25 | mL |

*Evaporates during processing.

Manufacturing Process:

1) 95% of the required quantity of water, cooled to −1° C. to −3° C., is placed in a mixing vessel that maintains the water temperature throughout the solution formation process.

2) Mannitol is added and stirred to dissolve.

3) The required quantity of azacitidine is added and the mixture is stirred continuously to form a solution.

4) The final volume is made up with water at −3° C. and stirred for about 5 minutes, until the solution is uniform.

5) The solution is filtered through a 0.2 μm sterilization filter.

6) The solution is filled into USP type I glass vials and loosely covered with a bromobutyl rubber or chlorobutyl rubber double slotted stopper.

7) The loosely stoppered vials are loaded into a lyophilizer with precooled shelves and lyophilized using the cycle described below, then the stoppers are fully seated and flip-off seals are attached.

| | | Shelf | | | | Cumulative |
|---|---|---|---|---|---|---|
| Step | | Temperature | Chamber Pressure | | Step Time | Time |
| No. | Step | (° C.) | (mBar) | (mTorr) | (minutes) | (minutes) |

Freezing

| | | | | | | |
|---|---|---|---|---|---|---|
| 1 | Hold | −5 | — | — | 2 | 2 |
| 2 | Rate | −25 | — | — | 15 | 17 |
| 3 | Rate | −40 | — | — | 75 | 92 |
| 4 | Rate | −42 | — | — | 50 | 142 |
| 5 | Hold | −40 | — | — | 75 | 217 |

Primary Drying

| | | | | | | |
|---|---|---|---|---|---|---|
| 8 | Hold | −40 | 0.37 | 278 | 60 | 277 |
| 9 | Rate | 20 | 0.37 | 278 | 1700 | 1977 |
| 10 | Rate | 30 | 0.250 | 188 | 60 | 2037 |
| 11 | Hold | 30 | 0.200 | 150 | 120 | 2157 |
| 12 | Rate | 35 | 0.200 | 150 | 300 | 2457 |
| 13 | Hold | 35 | 0.035 | 26 | 300 | 2757 |
| 14 | Rate | 40 | 0.035 | 26 | 120 | 2877 |

Secondary Drying

| | | | | | | |
|---|---|---|---|---|---|---|
| 15 | Hold | 40 | 0.0133 | 10 | 450 | 3327 |

Chemical stability is tested by storing the lyophilized vials under various conditions: 25° C. and 60% relative humidity (“A”); 40° C. and 75% relative humidity (“B”); and 60° C. (“C”); for one month. Impurity analyses are done before storage (“Initial”) and after storage, and are expressed as percentages of the label azacitidine content. Vials of a commercially available product (VIDAZA®) are similarly stored and analyzed.

| | Example 1 | | | | VIDAZA | |
|---|---|---|---|---|---|---|
| | | A | B | C | | B |
| Product | Initial | 1 Month | | | Initial | 1 Month |
| N-(formylamidino)-N′- | 0.38 | 0.05 | ND | ND | 3.03 | ND |
| β-D-ribofuranosyl | | | | | | |
| urea (RGU-CHO) | | | | | | |
| 1-β-D-ribofuranosyl- | 0.35 | 0.42 | 0.17 | 0.19 | 1.43 | 0.7 |
| 3-guanylurea (RGU) | | | | | | |
| Highest unidentified | 0.07 | 0.14 | 0.12 | 0.3 | 0.05 | 0.59 |
| impurity | | | | | | |
| Total Impurities, | 0.57 | 0.89 | 0.63 | 1.21 | 4.51 | 1.86 |
| excluding RGU-CHO | | | | | | |
| Water (mg/vial) | 1.71 | 1.14 | 1.04 | 1.36 | NA | NA |

NA = not analyzed.
ND = not detected.

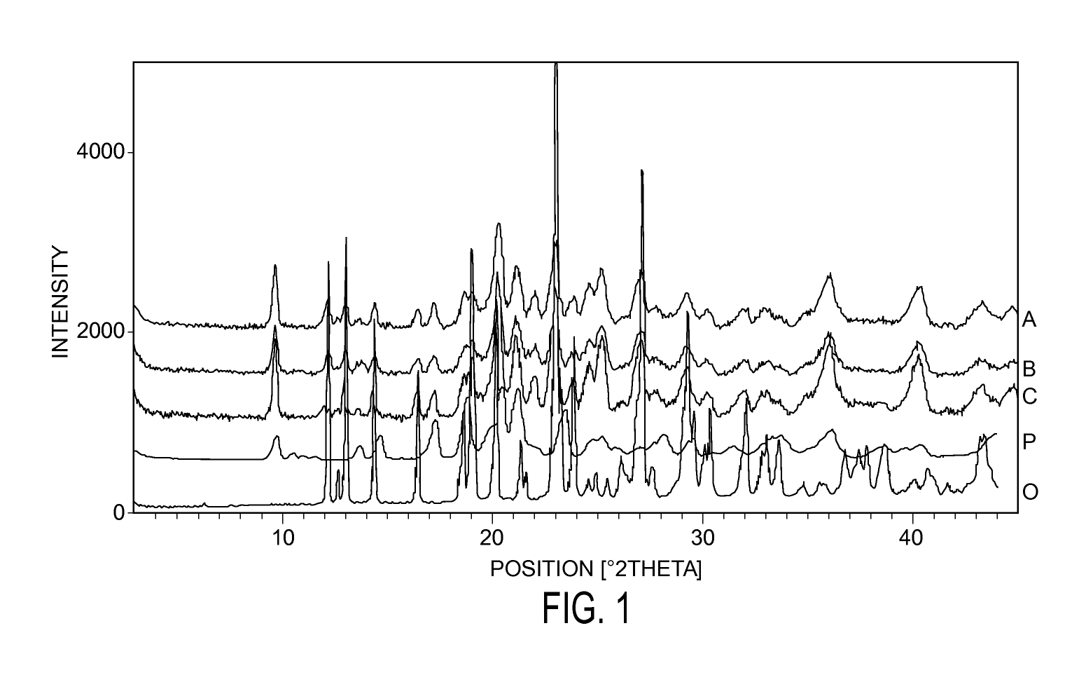

Physical stability is determined in an experiment where vials are stored. The product retains its physical form, indicated by powder X-ray diffraction patterns as shown in FIG. 1 of: the formulation prepared in Example 1 (“A”); a similarly prepared “placebo” formulation without the azacitidine ingredient (“P); the formulation after storage at 2-8° C. for 2 weeks (“B”); and the formulation after storage at 25° C. and 60% relative humidity for 2 weeks (“C”). Also shown is the powder X-ray diffraction pattern of the azacitidine ingredient (“D”). The powder X-ray diffraction patterns are generated using copper Kα radiation.

### Example 2

### Impurity Formation During Processing

Composition: similar to that of Example 1.

Manufacturing process for bulk solution: similar to steps 1-5 of Example 1.

The bulk solution is divided in two equal parts and stored for 5 hours at the temperatures in the table below:

| Part | Temperature | |
|---|---|---|
| 2A | −2° | C. |
| 2B | 2 to 8° | C. |

The stored solutions (25 mL quantities) are filled into type I glass vials and loosely covered, as in Example 1, are lyophilized using a similar procedure, and then are similarly stoppered and sealed.

Impurity analyses of the bulk solution as prepared, stored solutions, and the lyophilized products are tabulated below, where the values are percentages of the label azacitidine content.

| | | Stored Solution | | Lyophilized | |
|---|---|---|---|---|---|
| Impurity | Bulk | 2A | 2B | 2A | 2B |
| N-(formylamidino)-N′- | NA | NA | NA | 2.39 | 2.88 |
| β-D-ribofuranosyl urea | | | | | |
| (RGU-CHO) | | | | | |
| 1-β-D-ribofuranosyl-3- | 0.04 | 0.07 | 0.08 | 0.39 | 0.50 |
| guanylurea (RGU) | | | | | |
| Highest unidentified | 0.06 | 0.06 | 0.07 | 0.12 | 0.15 |
| impurity | | | | | |
| Total impurities, | 0.24 | 0.26 | 0.30 | 0.72 | 0.92 |
| excluding RGU-CHO | | | | | |

*NA: Not analyzed.

### Example 3

### Decitabine Pharmaceutical Formulation

| Ingredient | mg/Vial | |
|---|---|---|
| Decitabine | 50 | |
| Potassium dihydrogen phosphate | 68 | |
| Sodium hydroxide | 11.6 | |
| Water* | q.s. to 15 | mL |

*Evaporates during processing.

Manufacturing Process:

1) About 90% of the required quantity of water (cooled to −1° C. to −3° C.) is placed in a mixing vessel that maintains the water temperature throughout the solution formation process.

2) Monobasic potassium phosphate is added and stirred to dissolve.

3) Sodium hydroxide is added and stirred to dissolve.

4) Decitabine is added and the mixture is stirred continuously to form a solution.

5) The final volume is made up with water at −1° C. to −3° C. and stirred for about 10 minutes until the solution is uniform. The solution is stored at −1° C. to −3° C.

6) While maintaining the solution temperature at about −1° C. to −3° C., the solution is filtered through a 0.2 μm sterilization filter.

7) The solution is filled into USP type I glass vials and loosely closed with double slotted stoppers. At a temperature of about −1° C. to −3° C., the manufacturing process from addition of drug until loading into a lyophillizer should be completed within 3 hours.

8) The loosely stoppered vials are loaded into a lyophilizer with precooled shelves at about −15° C. and lyophilized using the cycle described in Example 1, then the stoppers are fully seated and flip-off seals are attached.

1. A pharmaceutical formulation for parenteral administration and comprising azacitidine, prepared by a process comprising:
a) preparing an aqueous solution containing azacitidine at about −3° C. to about −1° C.; and
b) lyophilizing the solution.

2. The pharmaceutical formulation of claim 1, wherein the process further comprises storing the solution below about 10° C., prior to lyophilizing.

3. The pharmaceutical formulation of claim 1, wherein the process further comprises storing the solution at about −3° C. to about −1° C., prior to lyophilizing.

4. The pharmaceutical formulation of claim 1, wherein azacitidine retains its physical form during storage in a closed container for at least two weeks at 25° C. and 60% relative humidity.

5. The pharmaceutical formulation of claim 1, wherein azacitidine retains its physical form during storage in a closed container for at least two weeks at 2 to 8° C.

6. The pharmaceutical formulation of claim 1, comprising less than about 1 percent of a 1-β-D-ribofuranosyl-3-guanylurea impurity, by weight of the label azacitidine content.

7. The pharmaceutical formulation of claim 1, comprising less than about 5 percent of a N-(formylamidino)N′-β-D-ribofuranosylurea impurity, by weight of the label azacitidine content.

8. The pharmaceutical formulation of claim 1, comprising less than about 2 percent of total drug-related impurities, excluding a N-(formylamidino)N′-β-D-ribofuranosylurea impurity, by weight of the label azacitidine content.

9. The pharmaceutical formulation of claim 1, in a lyophilized form that can be reconstituted, or in the form of a ready-to-use solution.

10. The pharmaceutical formulation of claim 1, in the form of a dry powder, solution, dispersion, emulsion, or suspension.

11. The pharmaceutical formulation of claim 1, having a water content less than about 6 percent by weight.

12. The pharmaceutical formulation of claim 1, wherein the formulation is packaged in a vial, ampoule, or a pre-filled syringe.

13. The pharmaceutical formulation of claim 1, in a kit comprising a container holding the formulation, a sterile reconstitution vehicle, and a sterile syringe.
